(19) United States
(12) Patent Application Publication
(10) Pub. No.: US 2017/0071590 A1
(43) Pub. Date: Mar. 16, 2017

(54) SUTURE ANCHOR

(71) Applicant: SUTURE ARMOR LLC, Marietta, GA (US)

(72) Inventor: W. Eric MACLEOD, Sea Island, GA (US)

(73) Assignee: SUTURE ARMOR LLC, Marietta, GA (US)

(21) Appl. No.: 15/054,245

(22) Filed: Feb. 26, 2016

Related U.S. Application Data
(60) Provisional application No. 62/216,775, filed on Sep. 10, 2015.
(60) Provisional application No. 62/289,543, filed on Feb. 1, 2016.

(51) Int. Cl. A61B 17/04

(52) U.S. Cl. CPC A61B 17/0401; A61B 2017/0414

(57) ABSTRACT

A suture anchor includes a generally circular body, a pair of wings, a filament, and center opening formed through the circular body. The circular body includes an outer diameter and an inner diameter bounding the center opening, and defines a generally vertical centerline and a vertical tangential plane. The wings generally extend beyond the vertical centerline to a distance approximate to, greater than, or less than the vertical tangential plane thereof. The filament is threaded at least once through a portion of the circular body. The circular anchor body forms a ring providing improved structural integrity and anchoring retention, and enables a lateral row surgical attachment technique.

### CROSS-REFERENCE TO RELATED APPLICATIONS

This application claims the priority benefit of U.S. Provisional Patent Application Ser. No. 62/216,775 filed Sep. 10, 2015 and of U.S. Provisional Patent Application Ser. No. 62/289,543 filed Feb. 1, 2016, the entireties of which are hereby incorporated herein by reference for all purposes.

### TECHNICAL FIELD

The present invention relates generally to the field of medical devices and surgical couplings and anchors, and more particularly to a suture anchor for anchoring filament to a bone for attachment of soft tissue to bone.

### BACKGROUND

Suture anchors are used for attachment of connective tissue to bone in orthopedic surgical procedures. Typically, the anchors of suture devices for permanent attachment to bone are formed from a metal or hard plastic material (e.g., aluminum or other metallic materials, PEEK, biocomposite), and can be quite large and may require a substantial amount of bone material to be removed such that the anchor and suture can be affixed thereto. Removing a substantial amount of bone for placement and securing the anchor and suture is typically undesirable, and metallic or hard plastic anchors may increase the likelihood of injury to a joint, for example, if the anchor becomes dislodged from the bone. In addition, bone preservation makes revision surgery easier.

Accordingly, it can be seen that needs exist for a new and improved suture anchor. It is to the provision of a suture anchor meeting these and other needs that the present invention is primarily directed.

### SUMMARY

In example embodiments, the present invention provides a suture anchor for permanent attachment to a bone, for example, for connecting connective tissue to a bone via a suture material or filament. In one aspect, the present invention relates to a suture anchor including a generally circular body, a pair of wings, a filament, and a central opening formed through a portion of the circular body. The circular body includes an outer diameter and an inner diameter, and defines a generally vertical centerline and a vertical tangential plane. A pair of wings optionally extend generally outwardly and oppositely from each other about portions of the circular body. The wings generally extend beyond the vertical centerline to a distance approximate to, greater than, or less than the vertical tangential plane thereof. The filament is threaded at least once through a portion of the circular body.

Optionally, an elongate placement member is provided for extending through an aperture in the anchor body or otherwise engaging a portion of the circular anchor body whereby directional and positional control of the anchor can be established to place the anchor as desired. In some example forms, the circular anchor body and the pair of wings are formed from a braided ultra high molecular weight polyethylene fiber, or any other material.

In another aspect, the invention relates to an anchoring device for permanently coupling connective tissue to bone. The anchoring device includes a circular element formed of a ring of braided fiber or an otherwise soft material having an outside diameter and a corresponding inside diameter of which the braided fiber or otherwise soft material having cross sectional properties which are significantly smaller than said major diameter. A continuation of the braided fiber or otherwise soft material extends from the circular element past its vertical centerline to a distance approximate to, greater than or less than a vertical tangential plane extending from its major diameter. A central opening is formed within a portion of the circular element. In example forms, the circular element has the ability for a filament to be passed through it in any and all directions to and from its center point.

In still another aspect, the invention relates to method of anchoring filament to a bone. The method includes providing a soft fiber loop comprising a pair of wings extending therefrom, filament threaded at least once through the loop, and a central opening formed through a portion of the loop; providing an elongated member comprising a coupling end having a pair of fingers extending therefrom; engaging the loop between the fingers of the elongated member; and establishing directional and positional control of the soft fiber loop to place the fiber loop into engagement with bone tissue as desired.

In yet another aspect, the invention relates to a method of anchoring filament to a bone. The method includes providing a circular anchor ring element; threading a filament material through at least a portion of the circular anchor ring element; providing a cylindrical element comprising a lumen extending therethrough; movably mounting the circular anchor ring element within the lumen of the cylindrical element; providing a longitudinal displacement member; movably mounting the displacement member within the cylindrical element for displacing the circular anchor ring element within the lumen; and establishing directional and positional control of the cylindrical element such that the circular anchor ring element movably mounted therein can be positioned and placed as desired.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description are exemplary and explanatory of preferred embodiments of the invention, and are not restrictive of the invention, as claimed.

### BRIEF DESCRIPTION OF THE DRAWINGS

FIG. 1 is a plan view of a circular anchor ring element of a suture anchor according to an example embodiment of the present invention.

FIG. 2 is a plan view of a suture anchor incorporating the anchor ring element of FIG. 1 and having wings extending from the anchor ring according to an example embodiment of the present invention.

FIG. 3 is a plan view of the winged circular suture anchor element of FIG. 2 with a suture filament threaded or passed through portions thereof, according to an example embodiment of the present invention.

FIG. 4 is a top perspective view of the suture anchor of FIG. 3, and showing an optional aperture formed through a portion of the circular anchor ring element.

FIG. 5 is a perspective view of a suture anchor engaged by an installation tool for placement of the anchor according to another example embodiment of the present invention.

FIG. 6 is a perspective view of a suture anchor element movably mounted within a placement tube and having filament threaded therethrough, and showing a longitudinal installation member movably mounted within the tube for displacing the suture anchor, according to another example embodiment of the present invention.

FIGS. 7-9 show front, side and perspective views of a winged circular suture anchor according to an example embodiment of the invention.

FIG. 10 shows a suture anchor in a neutral/relaxed configuration and abutting an opening within a bone whereby the suture anchor will be inserted therein and attached to the bone according to another example embodiment of the present invention.

FIG. 11 shows another example embodiment of a suture anchor engaged by an installation tool according to the present invention.

FIG. 12 shows another example embodiment of a suture anchor engaged by an installation tool according to the present invention.

FIG. 13 shows another example embodiment of a suture anchor according to the present invention.

FIGS. 14A, 14B and 14C show front, side and perspective views of a suture anchor having an alternate wing configuration, according to another example embodiment of the present invention.

FIG. 15 shows another example embodiment of a suture anchor according to the present invention.

FIG. 16 shows another example embodiment of a suture anchor and installation tool and placement method according to the present invention.

FIG. 17 shows an example embodiment of a lateral row suture anchor assembly according to the present invention.

FIG. 18 shows another example embodiment of a lateral row suture anchor according to the present invention.

FIGS. 19 and 20 show placement of lateral row and medial row anchors in a surgical procedure for reattachment of tendon to bone of the humeral head, according to an example embodiment of the invention.

### DETAILED DESCRIPTION OF EXAMPLE EMBODIMENTS

The present invention may be understood more readily by reference to the following detailed description taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

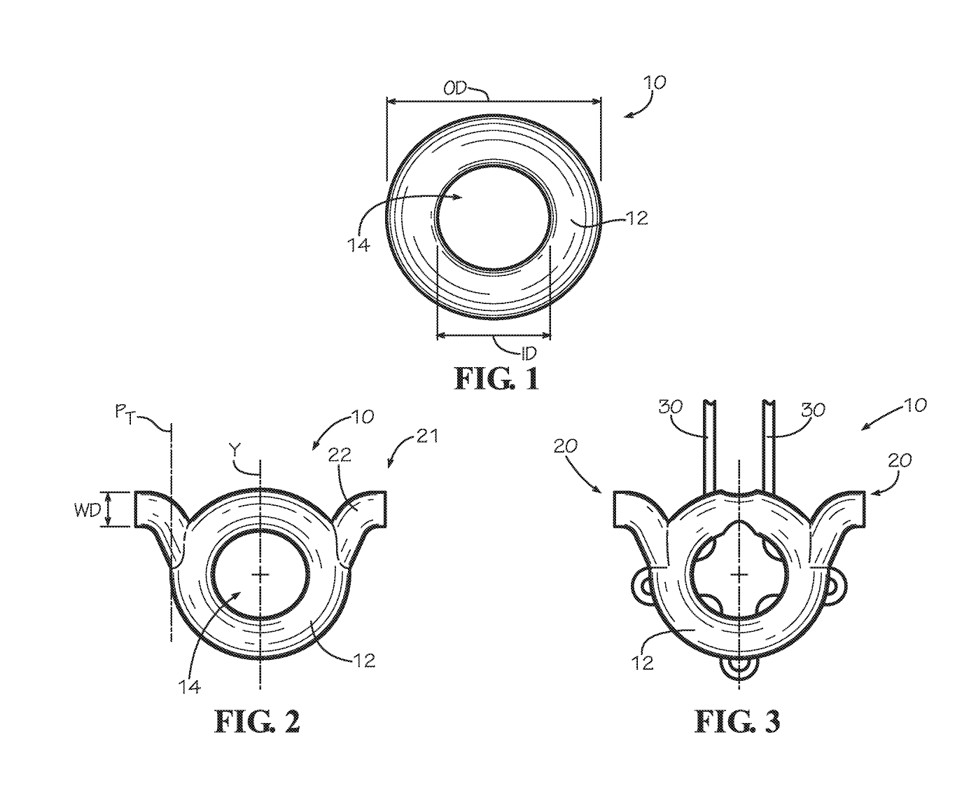

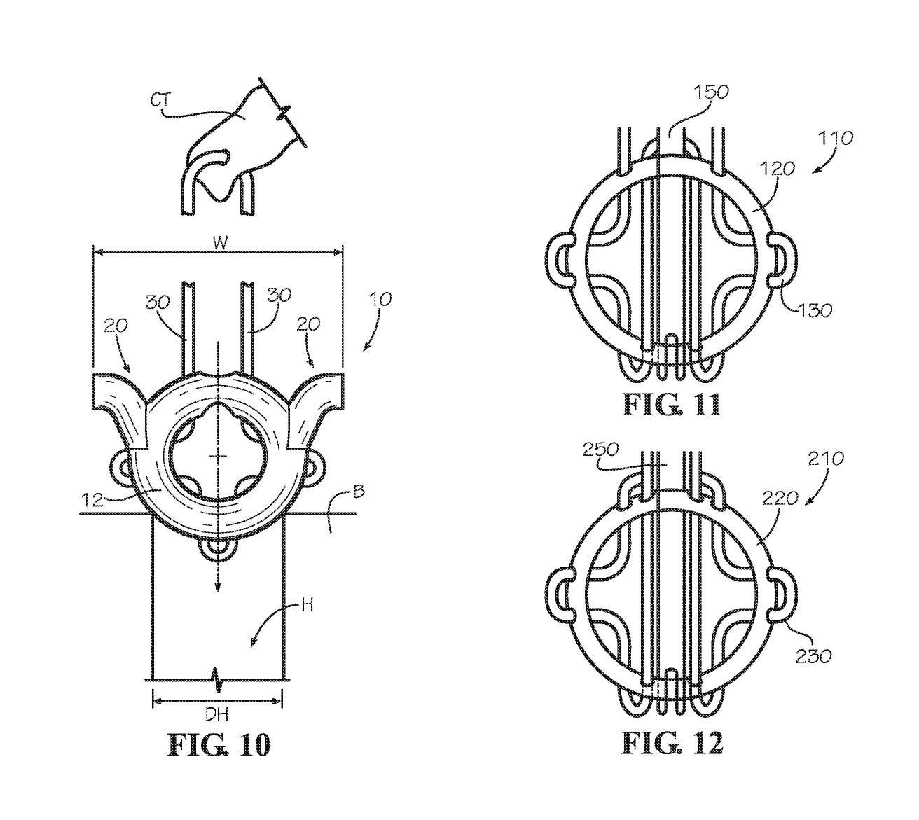

With reference now to the drawing figures, wherein like reference numbers represent corresponding parts throughout the several views, FIGS. 1-6 show a generally circular anchoring element or suture anchor 10 according to an example embodiment of the present invention. In example forms, the suture anchor 10 is utilized for anchoring suture filament(s) (as will be described below) to a bone B, for example, whereby the other end of the suture filament(s) anchor a tendon, a ligament, cartilage, muscle, fascia, or other connective or soft tissue for attachment and connection between the bone and tissue (see FIG. 10).

As depicted in FIGS. 1-2, the suture anchor 10 comprises a generally circular ring-like element, for example, generally having a continuous circular band 12 comprising an outer diameter OD and an inner diameter ID surrounding a central opening 14. In example forms, the band or ring 12 is generally formed from a braided fiber or an otherwise generally soft material. According to one example form, the band 12 is formed from a braided fiber comprising ultra-high molecular weight polyethylene (UHMW PE) strands. According to one example form, the fiber stands are in the form of Honeywell® Spectra fiber, which are braided using a Cortland 90326, Cortland 90259, or other braid pattern. In example embodiments, the ring 12 has an outside diameter OD of between 7-11 mm, more preferably between 8-10 mm, and for example about 9 mm; an inside diameter ID of between 5-8 mm, more preferably between 6-7 mm, and for example about 6.5 mm; and a thickness or cross-sectional diameter of 0.75-2.5 mm, more preferably between 1.0-1.5 mm, and for example about 1.25 mm. In additional embodiments, anchors of smaller or larger dimensions may be provided, for example, in applications directed to attachment to smaller or larger bone. Optionally, other materials, sizes and/or braiding patterns may be used as desired. In alternate example embodiments, the suture anchor 10 can comprise a bioresorbable suture material, PEEK, Teleflex, polyethylene, nylon, PLLA, PLGA, PCA, or any suture material. Preferably, the fiber strands are soft such that there is less potential for injury to joints, etc. if the suture anchor 10 was to become dislodged from the bone B. In example forms, the cross-section of the circular band 12 is generally circular, toroidal, donut-shaped, or tubular in shape. Optionally, other cross-sectional shapes (e.g., square, oval, polygonal, or other shapes) may be used according to other example embodiments.

According to alternative example embodiments of the present invention, the inner diameter ID is between about 4-8 millimeters (mm), more preferably about 6.2 mm, and the outer diameter OD is between about 5-12 mm, more preferably about 9 mm (see FIG. 7). Furthermore, the thickness or the diameter of the cross section T of the circular band 12 is between about 0.5-2 mm, or more preferably about 1.4 millimeters (see FIG. 8).

As depicted in FIG. 2, the circular band 12 optionally comprises one or more extension members or wings 20 extending from a portion of the circular band 12. For example, according to one example form, the circular band 12 generally comprises two outwardly and oppositely extending wings 20, which generally extend from portions of the band 12 past a vertical centerline Y to a distance approximate to, greater than, or less than a vertical tangential plane P_{T} extending from its outer diameter OD. According to example forms, the wings 20 are generally formed from a pair of braided fiber material segments 22 and comprise a substantially similar thickness or diameter WD as the band 12 thickness T, as shown in FIG. 8. According to some example forms, the braided fiber segments 22 are generally L-shaped or curved whereby the first ends thereof are braided with the band 12 and extend therefrom at an outward and upwards angle relative to the vertical centerline Y (about 25 degrees, see FIG. 7), and wherein a radiused transition is provided for directing the second ends thereof to extend oppositely along an axis generally perpendicular relative to the vertical centerline V and beyond the vertical tangential plane. Optionally, the extension of the wings 20 may be configured as desired, and wherein the extension thereof at least extends beyond the vertical centerline Y but may not extend beyond the vertical tangential plane. As similarly described above, the cross section of the wings 20 is generally circular comprising the diameter WD, which is generally between about 0.5-2.0 millimeters, more preferably about 1.4 millimeters. Optionally, the wings 20 may be formed from other cross-sectional shapes (e.g., square, oval, polygonal, or other shapes) as desired.

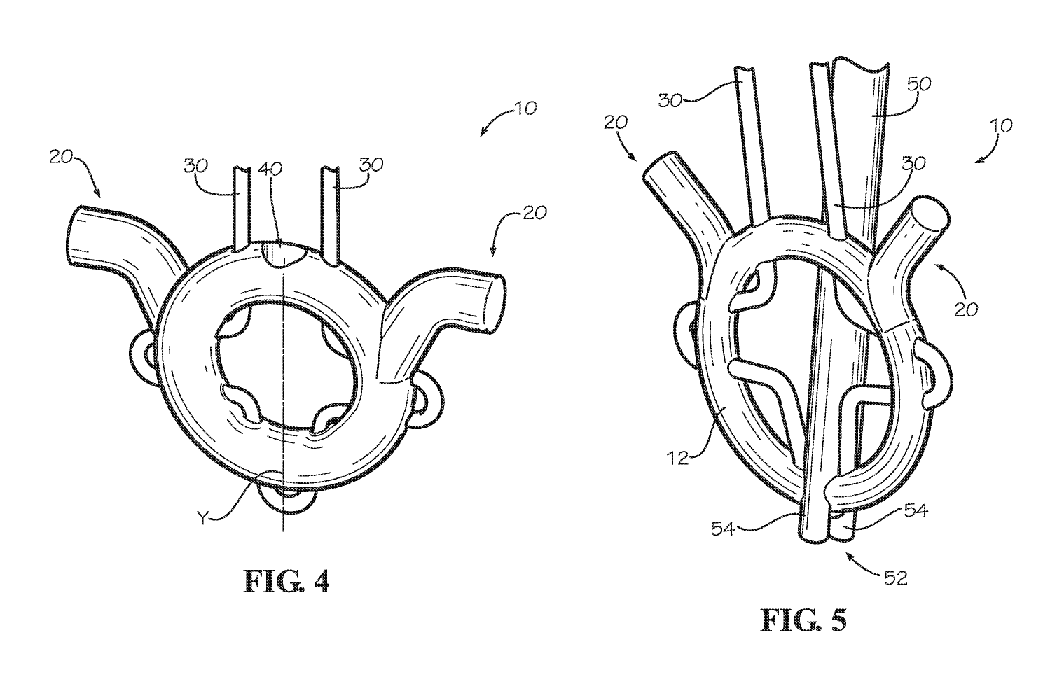

Preferably, as depicted in FIG. 3, the suture anchor 10 further comprises one or more pieces of suture filament or thread 30 extending through the circular body 12. According to one example form, the suture filament 30 is preferably threaded through the circular body 12 a plurality of times, for example, wherein the filament 30 passes through a portion of the circular body 12 about two times, for example about four times, and in other example embodiments about 8 times. In alternate embodiments, the suture filament 30 is threaded through the circular ring 12 more or fewer times, as suited to a desired application. According to example forms, the filament 30 generally comprises two free ends extending generally parallel with respect to each other and the vertical centerline Y, and wherein the filament 30 between the free ends is threaded through portions of the circular body, for example, from a position adjacent the outside diameter OD of the body 12, through the body 12 and to a position adjacent the inside diameter ID of the body 12. Furthermore, the filament 30 is then threaded back from a position adjacent the inside diameter ID, through the body 12, and then to a position adjacent the outside diameter OD. For example, the suture filament 30 may be looped through the anchor body ring 12 at diametrically opposed positions, at or about 90° increments about the circumference of the circular body ring in a cruciform pattern. For example, with reference to FIG. 4, the suture filament extends through a first top opening though the body ring 12 through the body ring and into the central opening of the ring, then loops out through a first left side opening and back in through a second left side opening, then loops back out through a first bottom opening and back in through a second bottom opening, then loops back out through a first right side opening and back in through a second right side opening, and finally back out through a second top opening in the body ring, in a generally cruciform pattern. According to example forms, the filament 30 is threaded through the circular body 12 about positions around the circular body 12 until the free ends of the filament 30 are generally positioned parallel relative to each other and extending away from the circular body 12. Preferably, the circular body 12 comprises the ability for the filament 30 to be able to be passed through it in any and all directions relative to and from its center point.

In example embodiments, the circular body or band 12 of the suture anchor 10 (with or without wing extensions 20) comprises an integral unitary body forming a complete 360° torus or toroid-shaped body having annular and circular cross-sections with aperture central opening 14. This circular structure provides improved overall structural rigidity, higher strength in compression, and increased bulk, relative to a U-shaped, V-shaped or partial-ring suture anchor configuration, resulting in increased effective anchoring diameter and improved resistance to pull-out failure. The center opening 14 of the ring also allows connection or anchoring of other anchors or objects to the band 12, for example in lateral row anchor techniques as described in greater detail below. In example embodiments, the anchor 10 comprises an “all-suture” soft suture anchor, wherein the band 12 (including wings 20 if present) comprises a larger diameter flexible suture material, and wherein the suture filament(s) 30 comprise a substantially smaller diameter flexible suture material.

As depicted in FIG. 4, an aperture 40 is optionally provided at or around a top portion of the circular body 12, which is generally axially aligned with the vertical centerline Y and positioned between the free ends of the filament 30. According to example forms, the aperture 40 is generally positioned about midway between the wings 20 and is configured for receiving an elongate installation member or tool, for example, which provides for inserting the suture anchor 10 within the bone B of a subject. According to example forms, the aperture is generally circular in shape. Optionally, other shapes (e.g., square, oval, polygonal, or other shapes) can be provided as desired. Alternatively, the placement aperture 40 is omitted, and the placement tool engages the body of the anchor ring 12, for example as described below.

FIG. 5 shows an example method of placement of the suture anchor 10, and a placement tool 50 for carrying out the method, according to example forms of the invention. In the depicted embodiment, the elongate installation tool 50 extends alongside the top portion of the circular body 12 and engages a lower portion thereof, to position the anchor at a target placement site, and to insert the anchor into a hole drilled in the bone at the placement site. In alternate embodiments, such as for example in an example method of placement of a suture anchor having an insertion aperture 40 as shown in FIG. 4, the insertion tool 50 can optionally be positioned through the aperture 40 and generally engages with a lower portion of the circular body 12 for positioning and placement of the anchor into a bone hole in similar fashion. According to example forms, the installation tool is substantially rigid and comprises a forked end 52 having a semi-circular recess with two generally finger-like elements 54 extending therefrom on opposite sides of the recess. Preferably, the finger-like elements 54 generally overlap or are positioned along sides of portions of the circular body 12 to hold the suture anchor 10 when the installation tool 50 is engaged with the circular body 12.

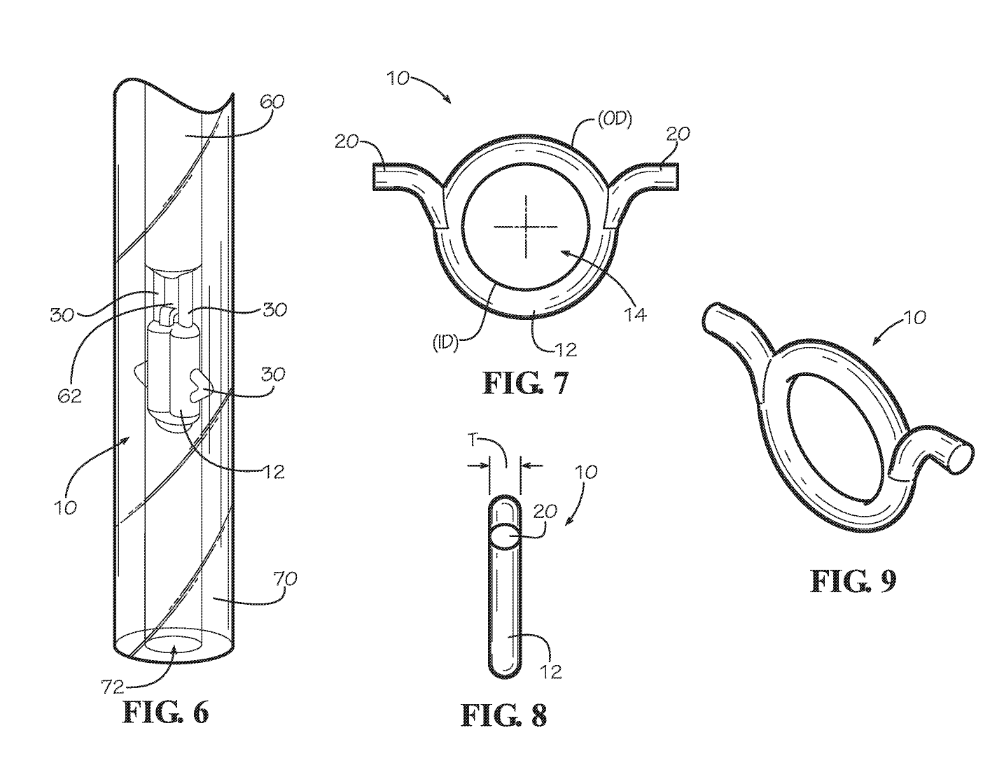

FIG. 6 shows an additional installation method or procedure according to another example embodiment of the present invention. According to example forms, the suture anchor 10 comprises the circular body 12 and filament 30 threaded therethrough, and the suture anchor 10 is movably mounted within a cylindrical member or tube 70 (e.g.; comprising a lumen 72 therein) such that a longitudinal member or rod 60 (comprising an end finger or manipulator 62) can be inserted within the lumen 72 for displacing the suture anchor 10. For example, according to an example embodiment, the present invention relates to a method of anchoring the filament 30 to the bone B, for example, by placing the circular body 12 into the aperture of the cylindrical rod 70, providing a longitudinal member 60 having a manipulator 62 extending therefrom for contacting and displacing the circular body 12 in an axial direction within the lumen 72, and positioning the cylindrical rod 70 as desired.

FIGS. 7-9 show the winged ring element of suture anchor 10 of FIG. 2 in greater detail. FIG. 10 shows the suture anchor 10 generally positioned adjacent an opening or pre-drilled hole H formed within a bone B and prior to receiving the elongate member 50 for insertion of the suture anchor 10 therein. According to example forms, the outer diameter OD of the cylindrical body 12 is about 1.2-1.5 times larger than the diameter DH of the drilled hole H, and the overall width W of the suture anchor 10 (e.g., between ends of the wings 20) is between about 1.5-2.5 times larger than the diameter DH of the drilled hole H. According to example forms, the overall width W defined between the wings 20 is between about 10-20 mm, more preferably about 15 millimeters. According to example forms, the diameter DH of the drilled hole H is between about 1.0-3.0 millimeters, more preferably about 1.5 millimeters. For example, about 14.3 mm³ of bone may be removed to form the drilled hole H.

Thus, when the suture anchor 10 is inserted within the bone hole H and the suture filaments 30 are pulled taut, the circular body 12 generally bunches up and deforms within the hole H for providing a solid anchoring hold therein. Preferably, the filament 30 extending out of the hole H and anchored to the circular body 12 is threaded to stitch the connective tissue CT such that the connective tissue is drawn into the hole and against the bone B, whereby the bone B and tissue heal and grow together to provide a substantially permanent connection therebetween. According to example forms, the wings 20 preferably provide additional frictional interference within the hole H and/or act as an abutment or stop against the compact bone at the periphery of an anchor site, and increase the anchor volume within the bone upon placement, for example, to provide increased anchoring attachment therein. According to example form, the wings 20 provide grip below the cortical layer (unshown) of bone B. Preferably, the circular body 12 in combination with the wings 20 and the filament 30 threaded therethrough provide a substantially high strength of fixation, for example, to resist one or more forces the body applies to the connective tissue CT to pull the suture anchor 10 out of the bone B. The presently claimed invention preferably impacts less of an area of a bone B as compared to other known suture anchors. For example, by providing a relatively small, soft, fibrous, and flexible anchor, anchoring of connective tissue to a bone can be less invasive, destructive and without removing a substantial about of bone or mass thereof.

According to another example embodiment, the present invention further relates to methods of anchoring filament to a bone. According to one example form, the method comprises providing a soft fiber loop or flexible ring comprising a pair of wings extending therefrom, filament at least threaded once through the loop, and aperture central opening formed through a portion of the loop; providing an elongated member comprising a coupling end having a pair of fingers extending therefrom; optionally inserting the elongated member through an installation aperture in the loop or ring, or placed alongside the loop or ring with the fingers engaging a section of the loop or ring; and establishing directional and positional control of the soft fiber loop to place the fiber loop as desired. According to another example form, the method comprises providing a circular element; threading a filament material through at least a portion of the circular element; providing a cylindrical element comprising a lumen extending therethrough; movably mounting the circular element within the lumen of the cylindrical element; providing a longitudinal displacement member; movably mounting the displacement member within the cylindrical element for displacing the circular element within the lumen; and establishing directional and positional control of the cylindrical element such that the circular element movably mounted therein can be positioned and placed as desired.

According to another example form, the present invention relates to a method in which the filament 30 is anchored to a bone B by placing an elongated member 50 through an optional aperture 40 of the circular body 12 until meeting the element opposite the aperture (a portion of the circular body 12) whereby directional and positional control of the circular body 12 can be established to place the circular body 12 as desired. Alternatively, the aperture is omitted from the anchor, and the method comprises placement of the elongate installation tool alongside a proximal portion of the anchor ring and into engagement with a distal portion of the anchor ring to position and place the anchor.

According to another example form, the present invention relates to a method of preparing an anchor site and installing the suture anchor 10. First, the proper anatomic location for soft tissue repair is visually or otherwise identified, for example arthroscopically or from an open approach. Next, the drill guide is placed onto the anatomic location on the bone that the anchor will be delivered. A drill bit of the appropriate size is then used to drill the guide hole. After the hole has been drilled in the bone, the drill bit will be removed from the cannulated drill guide. The anchor inserter with anchor 10 will then be placed into the drill cannula and malleted or pressed into the bone. Next, the sutures will be unraveled from the inserter handle and the inserter removed leaving the anchor 10 deep in the bone. Afterwards, the drill guide will be removed and the anchor 10 will be ‘seated’ into position with a slight tug of the sutures. The sutures will then be passed, in the configuration of preference, using an arthroscopic suture passer or a free needle through the identified soft tissue that could be either tendon, ligament, or cartilage. The suture will then be tied using knot of preference to complete the anatomic repair.

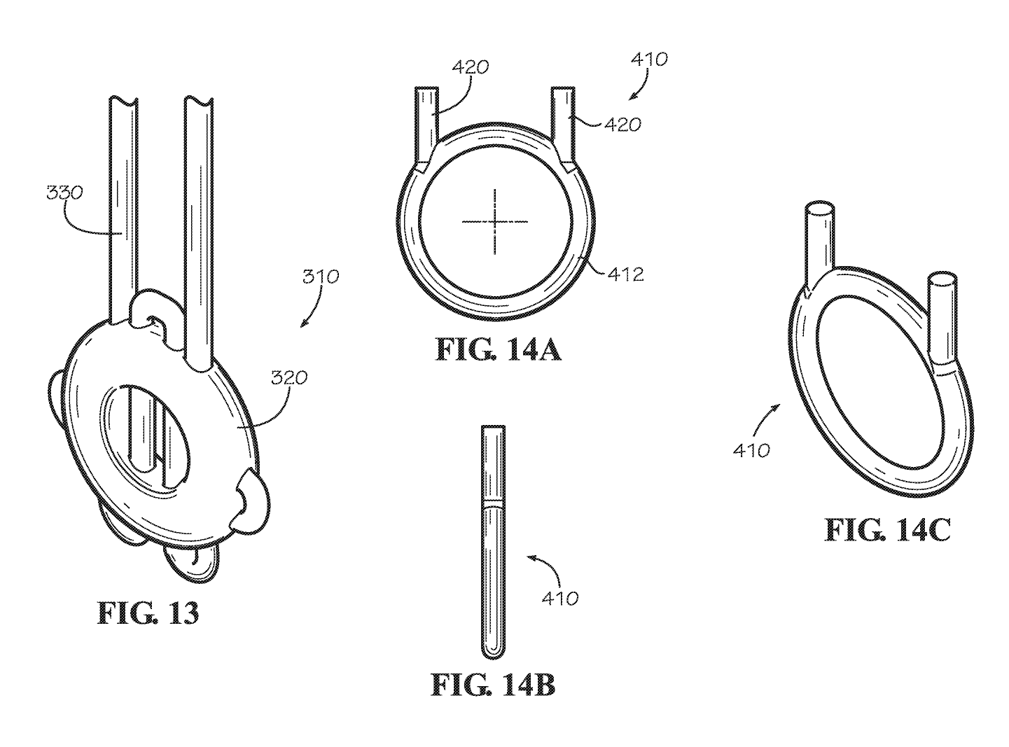

FIGS. 11 and 12 show alternate embodiments of suture anchors 110, 210 according to example forms of the invention, with differing filament weave patterns 130, 230 through the circular anchor body 120, 220, and an installation tool 150, 250 engaged with a lower portion of the anchor body. FIG. 13 shows another alternate embodiment of a suture anchor 310, with a circular anchor body or ring 320 and an interwoven suture filament 330.

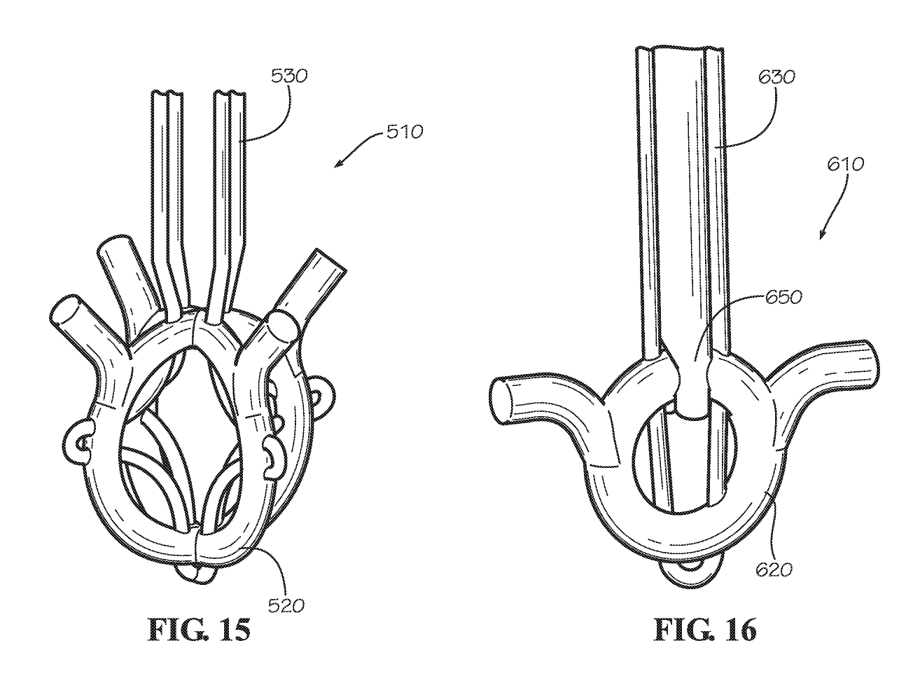

FIGS. 14A, 14B and 14C show front, side and perspective views, respectively, of a suture anchor 410 according to another example embodiment of the invention. The anchor 410 comprises an anchor body forming a ring or loop 412 of flexible, larger diameter suture material, and first and second vertical wings 420 extending outwardly and generally tangentially from the anchor body, generally parallel to the vertical centerline or axis of the anchor. Optionally, one or more filaments of flexible, smaller diameter suture material (unshown) is/are placed through the anchor body at one or more locations about the ring 412, for example in the manner of one or more of the above-described embodiments, to engage the anchor within a bone opening at the placement site upon tensioning of the filaments.

FIG. 15 shows a suture anchor 510 according to another example embodiment. In this embodiment, the anchor 510 comprises a “rescue” anchor capable of engagement in multiple dimensions. In example applications, the rescue anchor 510 is adapted for anchoring larger tears or in procedures where other anchor types may otherwise be inadequate. The suture anchor 510 comprises a three-dimensional anchor body 520 formed of an intersecting pair of generally circular rings oriented at right angles to one another to form a generally spherical or spheroid body. One or more suture filaments 530 is/are interwoven through one or both rings of the body 520, such that tensioning the filaments compresses the body for secure anchoring to bone or other tissue.

FIG. 16 shows another alternate embodiment of a suture anchor 610 and installation method, the suture anchor comprising a ring-shaped body 620 having opposed transversely extending horizontal wings, with a suture filament 630 interwoven through the body. A forked insertion tool 650 engages an upper portion of the body 620 between insertion points of the suture filament 630, for placement of the anchor 610 into a suture aperture formed in bone or other tissue to which the anchor is applied.

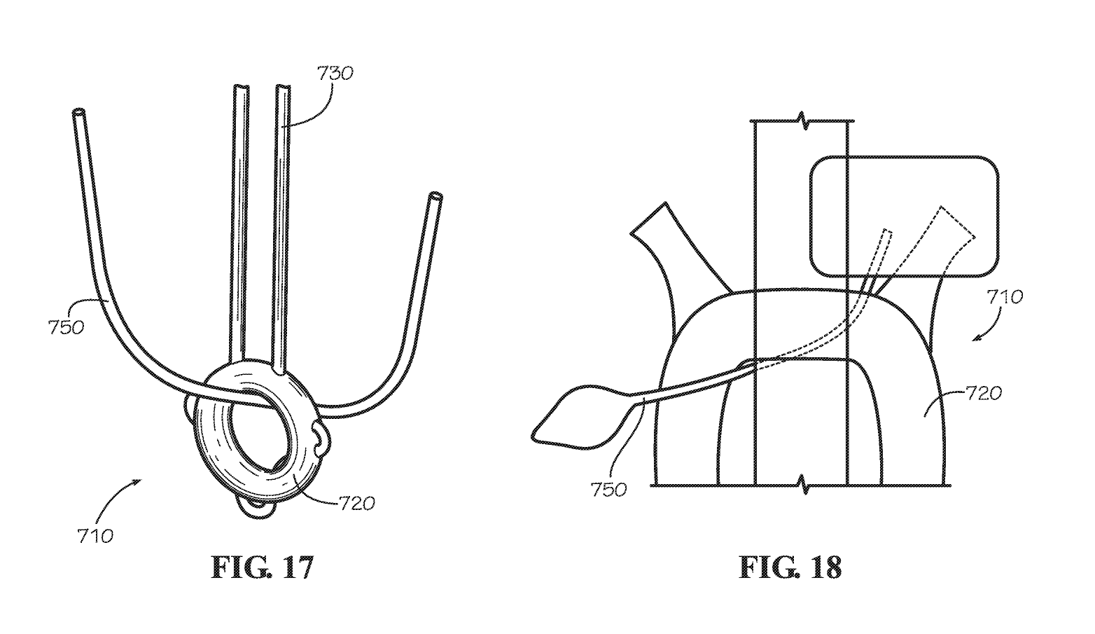

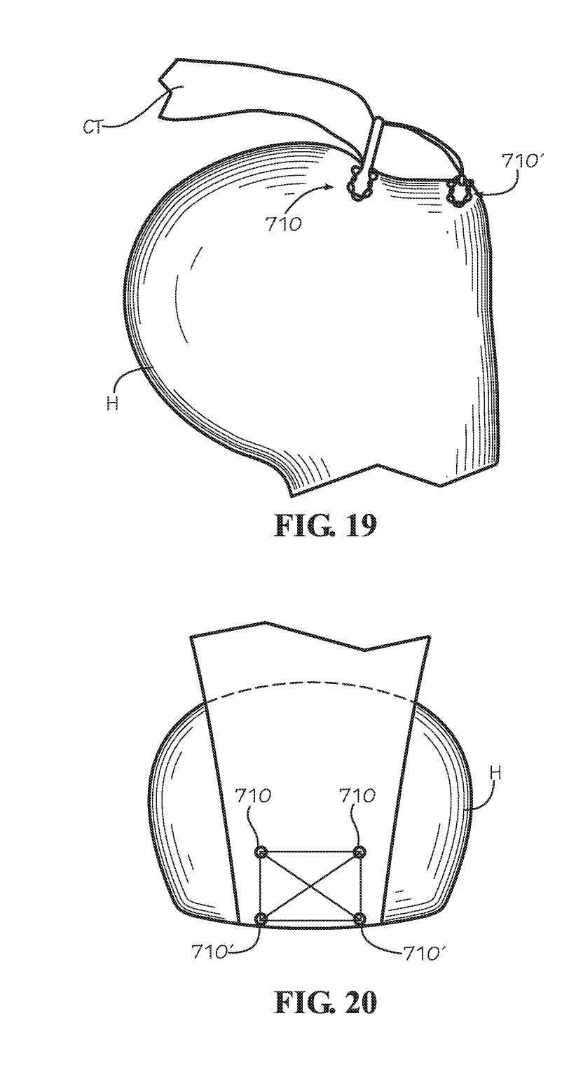

FIGS. 17 and 18 show an all-suture surgical suture anchor 710 adapted for application in a lateral row surgical technique shown in example form in FIGS. 19 and 20, for example to attach tendon or other connective tissue CT to a humeral head H or other bone tissue, according to example forms of the invention. After completion of the medial row fixation using medial row anchors 710, the uncut medial row sutures are brought outside the body and one is passed through the center or inner diameter of the lateral row anchor 710′ and through a wire loop 750. An appropriate sized hole is placed for lateral row fixation to the humerus H and the lateral row anchor is inserted into the hole and seated in place. The lateral row anchor sutures 710 can then be used to provide interrupted stitches into the rotator cuff or simply tied to one another to complete the fixation of the anchor. Next, the medial row suture that was passed through the lateral row anchor 710′ and the medial row suture that was not passed through the lateral row anchor 710′ are tied together to complete the connection between the medial and lateral row anchors. Depending on the size of the tear and the number of medial row anchors 710, multiple configurations may be used to connect the medial and lateral rows. For instance, multiple sutures may be passed through the center of the lateral row anchor 710′ if needed. The ringed design of the anchor body allows sutures to be passed through the center opening of the ring and tied adjacently. The ringed design of the anchor body enables an all suture lateral row anchor, and also creates an entire rotator cuff repair system, whereby in combination with an all suture labial anchor, one has an entire shoulder repair system.

While the invention has been described with reference to preferred and example embodiments, it will be understood by those skilled in the art that a variety of modifications, additions and deletions are within the scope of the invention, as defined by the following claims.

What is claimed is:

1. A suture anchor comprising:
a flexible anchoring loop comprising a generally circular ring of flexible material having an outer diameter and defining a central opening having an inner diameter, the flexible material having a first thickness; and
a suture filament threaded through the flexible anchoring loop, the suture filament having a second thickness less than the first thickness.

2. The suture anchor of claim 1, further comprising a pair of wings extending from the anchoring loop.

3. The suture anchor of claim 1, wherein the anchoring loop comprises a braided suture material.

4. The suture anchor of claim 1, wherein the suture filament is threaded through the anchoring loop at a plurality points spaced about the anchoring loop.

5. The suture anchor of claim 1, wherein the suture filament is threaded through the anchoring loop a plurality of times at two or more spaced apart locations about the generally circular ring.

6. A suture anchor comprising:
a generally circular body comprising an outer diameter and an inner diameter, the circular body defining a generally vertical centerline and a vertical tangential plane;
a pair of wings generally extending outwardly and oppositely from each other about portions of the circular body, the wings generally extending beyond the vertical centerline to a distance approximate to, greater than, or less than the vertical tangential plane thereof; and
a filament threaded at least once through a portion of the circular body.

7. The suture anchor of claim 6, further comprising an aperture formed through a portion of the circular body.

8. The suture anchor of claim 7, further comprising an elongate member for extending through the aperture and coupling to a portion of the circular body whereby directional and positional control of the circular element can be established to place the circular element as desired.

9. The suture anchor of claim 6, wherein the circular body and the pair of wings are formed from a braided ultra high molecular weight polyethylene fiber.

10. The suture anchor of claim 6, comprising a bioresorbable material.

11. A method of anchoring filament to a bone comprising:
providing an anchor comprising a generally circular ring of flexible material having an outer diameter and defining a central opening having an inner diameter, and a filament threaded at least once through the generally circular ring of flexible material;
providing an elongated placement tool comprising a coupling end having a pair of fingers extending therefrom;
engaging the anchor with the fingers of the placement tool; and
establishing directional and positional control of the anchor with the placement tool to place the anchor at an anchoring site.

12. The method of claim 11, wherein the anchor comprises a pair of wings extending from the generally circular ring of flexible material, the wings engaging within the anchoring site upon placement of the anchor.

13. The method of claim 11, wherein the anchor comprises an aperture through the generally circular ring of flexible material, and wherein the placement tool is inserted through the aperture to place the anchor.

14. A method of anchoring filament to a bone comprising:
providing a an anchor comprising a generally circular anchor body element, and a filament material threaded through at least a portion of the circular element;
providing a cylindrical installation tool comprising a lumen extending therethrough;
movably mounting the anchor within the lumen of the cylindrical installation tool;
actuating a longitudinal displacement member through the lumen of the cylindrical installation tool to position and place the anchor at an anchoring site.

15. An all-suture surgical suture anchor comprising:
an anchor body comprising a complete ring of flexible material;
at least one suture filament interwoven through at least a portion of the anchor body.

16. A rescue anchor comprising a three-dimensional anchor body comprising at least one ring of flexible anchor material, and at least one suture filament at least partially embedded in the three-dimensional anchor body.

17. The suture anchor of claim 14, wherein the three-dimensional anchor body comprises a pair of interconnected rings.

18. A lateral row surgical anchoring technique comprising:
medial row fixation of soft tissue to bone with at least one medial row anchor, each medial row anchor comprising a medial row anchor body and at least one medial row suture filament;
passing the suture filament of a medial row anchor through an inner diameter of a lateral row anchor, the lateral row anchor comprising a ring shaped lateral row anchor body defining the inner diameter and at least one lateral row suture filament;
anchoring the ring shaped anchor body of the lateral row anchor to bone;
stitching soft tissue with the lateral row suture filament; and
tying the medial row suture filament to connect the medial row anchor and the lateral row anchor.

19. The lateral row surgical anchoring technique of claim 16, wherein the bone is the humerus, and the soft tissue is rotator cuff or labral tissue of a shoulder.

20. An all-suture anchor configured for use as the medial row anchor and/or the lateral row anchor in the anchoring technique of claim 16.
